(19) United States
(12) Patent Application Publication
(10) Pub. No.: US 2011/0305710 A1
(43) Pub. Date: Dec. 15, 2011

(54) NOVEL COMPOUNDS FOR THE TREATMENT OF GLAUCOMA OR OCULAR HYPERTENSION

(76) Inventor: Muhammad Abdulrazik, East-Jerusalem (IL)

(21) Appl. No.: 13/107,874

(22) Filed: May 14, 2011

(30) Foreign Application Priority Data
Feb. 28, 2010 (IL) PCT/IL2010/000166
May 13, 2010 (IL) 205774

(51) Int. Cl. A61K 38/18; A61K 38/28; A61P 27/06; A61K 31/7088; C07H 21/00; A61K 39/395; C07K 14/475; A61K 38/30

(52) U.S. Cl. 424/172.1; 514/8.1; 530/399; 514/6.5; 514/8.5; 514/44 R; 536/23.51

(57) ABSTRACT

This invention relates to compositions and methods for the treatment or prophylaxis of glaucoma and ocular hypertension, and for lowering of intraocular pressure. The invention provide VEGFR-1 agonists and combinations of vascular endothelial growth factor B (VEGF-B) with placenta growth factor (PLGF), ‘ Secreted protein acidic cysteine-rich’ (SPARC) antagonists, insulin, insulin like growth factor, their isoforms, their analoges, their gene-engineered modifications. Furthermore, the formentioned compositions and combinations are provided for coadministration with antiglaucoma agents, currently in clinical use, for the treatment or prophylaxis of glaucoma and ocular hypertension, for lowering of intraocular pressure, for increasing success rate of surgical procedures for the treatment or prophylaxis of glaucoma, and for preserving retinal ganglion cells.

This application is a continuation-in-part of PCT/IL2010/000166, now pending an US designated. The application also claims the priority date of the Israeli application No. 205774 filed on May 13, 2010.

### BACKGROUND OF THE INVENTION

Glaucoma is one of the worldwide leading causes of blindness. According to recent estimates, about 50 million people worldwide suffer from glaucoma while several millions of new glaucoma cases are expected each year. Glaucoma is a silent disease that may run undetected for many years to be diagnosed only at advanced stages with considerable damage to retinal ganglion cells and nerve fiber layer of the retina. (see: Glaucoma. In Basic and Clinical Science Course Section 10; American Academy of Ophthalmology, 2007-2008 ed.). Many pharmacological and surgical interventions have been used to slow down the progression of glaucoma but none of these interventions could cure or even arrest the progression of this neurodegenerative disease that could deteriorate to severe loss of vision and blindness. The glaucomas are generally divided into open-angle and closed-angle, primary and secondary, and further classified into acute and chronic forms. More than 80% of all glaucomas are chronic open angle glaucoma cases (COAG). Elevated intraocular pressure (IOP) is a long appreciated major risk factor for glaucoma. However, cumulating evidence from recent decades strongly suggests the contribution of mechanisms independent of intraocular pressure in the pathogenesis of this neurodegenerative disease. So far, the available interventions can treat only one single risk factor of glaucoma, the elevated intraocular pressure. Current pharmacologic interventions for the treatment of glaucoma include many classes of drugs, all designed to lower IOP by either decreasing aqueous production rate or increasing aqueous outflow or a combination of both mechanisms. Antiglaucoma drug classes that are approved for clinical use include prostaglandin analogues, beta-blockers, alpha2-adrenergic agonists, carbonic anhydrase inhibitors, miotics (direct cholinergic agonists and cholinesterase inhibitors) and nonselective adrenergic agonists (epinephrine and its derivatives) (See: 2006 Physicians' Desk Reference (PDR) for Ophthalmic medicines). Established antiglaucoma drugs that decrease aqueous production rate include nonselective beta adrenergic antagonists, selective beta-1 adrenergic antagonists, nonselective adrenergic agonists, alpha-2 adrenergic agonists and carbonic anhydrase inhibitors. Many other compounds have been reported to decrease aqueous humor production rate but are not approved for clinical use. Examples are calcium channel antagonists (verapamil, nifedipine), nitrovasodilators, atrial natriuretic factor, serotonergic receptor (5-HT1A) antagonists (ketanserin), angiotensin converting enzyme (ACE) inhibitors, H1-antihistamines (antazoline, pyrilamine), delta 9-tetrahydrocannabinol (a component of marijuana), cardiac glycosides (ouabain, digoxin), cyclic GMP (8-Bromo cyclic GMP). Established antiglaucoma drugs that increase aqueous outflow rate include nonselective adrenergic agonists, alpha-2 adrenergic agonists (increase uveal outflow), miotics (increase trabecular outflow) and prostaglandin analogues (increase uveal outflow). Experimental compounds that have been reported to increase aqueous humor outflow rate include hyaluronidase, proteases, matrix metalloproteinases, transforming growth factor-beta, cytochalasin B, cytochalasin D, ethacrynic acid, tienilic acid, staurosporine, latrunculins, ethyleneglycol bis (aminoethylether) tetraacetate (EGTA) and EDTA. However, these compounds are not approved for clinical use. While some of the currently available pharmacologic interventions in clinical ophthalmology can provide a significant lowering of intraocular pressure, none of these agents could cure or even arrest the progression of glaucomas. In cases were profound manipulations of the trabecular outflow are indicated, such goal is currently achievable only by surgical interventions.

### SUMMARY OF THE INVENTION

An object of the present invention is to provide novel compositions for the prophylaxis, slowing, halting or reversal of damage to ocular tissues and cells related to glaucoma and ocular hypertension.

The inventor of the present invention have earned to a surprising finding that VEGFR-1 agonists, SPARC antagonists, insulin growth factor, and euglycemic levels of insulin have a substantial effect in the prophylaxis, slowing, halting or reversal of damage to ocular tissues and cells related to glaucoma and ocular hypertension, which was additive when those peptides were coadministered as a combination treatment. Subsequently an extensive study was carried out with a view to clarify the correlation of those peptides with intraocular pressure and glaucoma prophylaxis and treatment. VEGFR-1 agonists, SPARC antagonists, insulin growth factor, and euglycemic levels of insulin have shown substantial effects as stand alones and additive effects as combination therapy, in the prophylaxis, slowing, halting or reversal of damage to ocular tissues and cells related to glaucoma and ocular hypertension, when administered to the eye in the invented method. The present invention has been made on the basis of the above findings, and the summary of the present invention is as follows.

The invention provides synergestic combination of peptides that possess anti-glaucoma and/or ocular hypotensive properties, where those pepides are selected from VEGFR-1 agonists, SPARC antagonists, euglycemic-insulin, insulin like growth factor, their isoforms, their analoges, their gene-engineered modifications, and combinations thereof for the prophylaxis, slowing, halting or reversal of glaucoma related damage to ocular tissues and cells, for the prophylaxis, slowing, halting or reversal of ocular hypertension related damage to ocular tissues and cells, for increasing success rate of surgical procedures for the treatment or prevention of glaucoma, and for the prophylaxis, slowing, halting or reversal of damage to retinal ganglion cells.

The attached claims define further embodiments of the present invention.

### DETAILED DESCRIPTION OF THE INVENTION

The invention relates to stimulating VEGFR-1 for prophylaxis, slowing, halting or reversal of glaucoma related damage to ocular tissues and cells, for the prophylaxis, slowing, halting or reversal of ocular hypertension related damage to ocular tissues and cells. The invention provides synergestic combination of peptides that possess anti-glaucoma and/or ocular hypotensive properties, where those pepides are selected from VEGFR-1 agonists, SPARC antagonists, euglycemic-insulin, insulin like growth factor, their isoforms, their analoges, their gene-engineered modifications, and combinations thereof for the prophylaxis, slowing, halting or reversal of glaucoma related damage to ocular tissues and cells, for the prophylaxis, slowing, halting or reversal of ocular hypertension related damage to ocular tissues and cells, for increasing success rate of surgical procedures for the treatment or prevention of glaucoma, and for the prophylaxis, slowing, halting or reversal of damage to retinal ganglion cells.

SPARC (secreted protein acidic and rich in cysteine) is a multifunctional glycoprotein that belongs to matricellular group of proteins. Reportedly, SPARC interacts with VEGFR-1 and have been shown to act by suppression of signaling through this receptor. Vascular endothelial growth factor B (VEGF-B; VEGF-B.sub.167, and VEGF-B.sub.186) and Placenta Growth Factor (PlGF-1, PlGF-2, PlGF-3) are members of the VEGF family that are pure agonists of VEGFR-1.

As used herein, the term “antibody” refers to an immunoglobulin molecule with a specific amino acid sequence evoked by an antigen, e.g., SPARC, Ccl-2, HO-1, SHP-1 or Ccr-2, and characterized by reacting specifically with the antigen in some demonstrable way. The term “antibody” encompasses polyclonal and monoclonal antibody preparations, CDR-grafted antibody preparations, as well as preparations including hybrid antibodies, altered antibodies, F(AB)′.sub.2 fragments, F(AB) molecules, Fv fragments, single domain antibodies, chimeric antibodies and functional fragments thereof which exhibit immunological binding properties of the parent antibody molecule. The antibodies can also be humanized.

As used herein, the terms “bind” or “binds” or “binding” means any interaction, whether via direct or indirect means, which affects a specified protein, receptor or protein/receptor subunit.

As used herein, the term “carrier” refers to a diluent, adjuvant, excipient, or vehicle with which the compositions of the present invention are administered. The term “pharmaceutically acceptable carrier” refers to a carrier that may be administered to a subject, and which does not destroy the pharmacological activity thereof and is nontoxic when administered in doses sufficient to deliver a therapeutic amount of the compound.

As used herein, “compound” refers to any agent, chemical substance, or substrate, whether organic or inorganic, or any protein including antibodies and functional fragments thereof, peptides, polypeptides, peptoids, and the like.

As used herein, the terms “antagonist” “inhibit(s)” or “downregulate” mean any decrease or reduction in functionality or activity.

As used herein, the terms “interact(s)” or “interacting with” or “interaction with” mean any reactive affect on a specified protein, receptor or protein/receptor subunit by another molecule or subunit, whether through binding, in the whole or in part (e.g., covalent, non-covalent, hydrogen) or signaling, regardless of the affect (e.g., antagonize, inhibit, downregulate, deactivate, interfere, agonize, promote, upregulate, neutralize).

As used herein, the term “introducing” means any means of delivery or administration, whether in vivo or in vitro, including simple contact.

The term “monoclonal antibody” is not limited to antibodies produced through hybridoma technology. The term “monoclonal antibody” refers to an antibody or functional fragment thereof that is derived from a single clone, including any eukaryotic, prokaryotic, or phage clone, and not the method by which it is produced. As used herein, the term “aptamer” means DNA or RNA molecules that have been selected from random pools based on their ability to bind other molecules, like nucleic acid, proteins, small organic compounds, and even entire organisms.

“therapeutically effective euglycemic amount of insulin” and “euglycemic insulin” administration in the present invention refers to the administration of insulin or insulin substitute in amounts and in a way that achieves the goal of lowering intraocular pressure and treating or preventing glaucoma and ocular hypertension while avoiding significant effects on the blood glucose levels and the induction of hypoglycemia.

Frier et al teaches that hypoglycemia can lower intraocular pressure through “profound autonomic discharge provoked by acute hypoglycaemia” (Frier et al., Br. Med. J. (Clin. Res. Ed.) 7 March 1987, Vol. 294, No. 6572, page 611, left column, 3^{′d} paragraph [comment], line 6). Similarly, Backon et al. (Br. J. Oph., 1990, vol. 74, pages 607-609) have used selective functional depression of the parasympathetic autonomic discharge to lower intraocular pressure in normal and glaucoma eyes.

The current invention have shown for the first time that Insulin can lower intraocular pressure not through the induction of hypoglycemia (euglycemic effect of insulin) but rather through direct effects of insulin on target ocular tissues. For example, please note Example No. 7 on page 36 of the original application: “Effect on Permeability”. In contrary, the prior art (Frier et al) teaches lowering of the intraocular only through the “hypoglycemic effect”. Koivikko et al. (Diabetes, vol. 54, March 2005, pages 744-750) provides support to this distinction between “euglycemic insulin” and “hypoglycemic insulin” interventions and their effects on the autonomic system. Koivikko et al. studied the effect of hypoglycemic and euglycemic insulin clumps on the vagal indexes (parasystemic part of the autonomic system) and came to conclusion that “the observed changes in vagal indexes during the hypoglycemic clamp were caused by hypoglycemia itself but not caused by hyperinsulinemia” (page 748, right column, 2^{″d} paragraph [Effect of Hyperinsulinemia], lines 11-13).

In summary, both Frier et al. and Backon et al. teach that the reduction of intraocular pressure is mediated through the autonomic system, namely by vagal depression, and Koivikko et al. teaches that hypoglycemia, but not insulin, can cause vagal depression,

In conclusion, lowering intraocular pressure by “euglycemic insulin” intervention means that this goal is achieved by direct effect of insulin on target ocular tissues and not through the indirect effect of hypoglycemia on intraocular pressure through the autonomic system.

### Definitions

VEGF-B: Vascular endothelial growth factor-B (VEGF-B) is a growth factor that belongs to the vascular endothelial growth factor family that, in humans, is encoded by the VEGF-B gene. In contrast to VEGF-A, VEGF-B plays a less pronounced role in the vascular system and VEGF-B exerts its effects via the Vascular endothelial growth factor receptor-1 (FLT1, or VEGFR-1 receptor).

Placenta Growth Factor-1: Placenta Growth Factor-1 (PlGF-1) is a member of the VEGF family that is a pure agonist of VEGFR-1.

SPARC: SPARC (secreted protein acidic and rich in cysteine) is a multifunctional glycoprotein that belongs to matricellular group of proteins. Reportedly, SPARC interacts with VEGFR-1 and have been shown to act by suppression of signaling through this receptor.

Agonists and antagonists: Agonist drugs activate receptors to produce the desired response. Conventional agonists increase the proportion of activated receptors. Inverse agonists stabilize the receptor in its inactive conformation and act similarly to competitive antagonists Antagonists prevent receptor activation. Preventing activation has many effects. Antagonist drugs increase cellular function if they block the action of a substance that normally decreases cellular function. Antagonist drugs decrease cellular function if they block the action of a substance that normally increases cellular function. Receptor antagonists can be classified as reversible or irreversible. Reversible antagonists readily dissociate from their receptor; irreversible antagonists form a stable, permanent or nearly permanent chemical bond with their receptor. Pseudo-irreversible antagonists slowly dissociate from their receptor.

Co-administration: coadministration of two or more drugs could be simultaneous, or by administration of one or more drugs before or after the administration of main drug. Simultaneous co-administration could be through one formulation or in separate formulations. Simultaneous co-administration via separate formulations could be done through same delivery route or via different administration routes.

Polypeptides and Polynucleotides: Polypeptides and Polynucleotides are respectively short and long polymers of amino acids.

siRNA: RNA interference (RNAi) is a system within living cells that takes part in controlling which genes are active and how active they are. Two types of small RNA molecules—microRNA (miRNA) and small interfering RNA (siRNA)—are central to RNA interference. RNAs are the direct products of genes, and these small RNAs can bind to other specific RNAs (mRNA) and either increase or decrease their activity.

“intraocular pressure ” in the present invention refers to measurement of the pressure of the fluid that fills the eye globe, the aqueous humor, which is determined by the interplay between the rate of aqueous humor production inside the eye and the resistance to aqueous outflow as it exits the eye through the anterior chamber angle towards the schlemm's canal. In the human eye, the rate of aqueous formation is 2.5 μL/minute while that in the rabbit eye is approximately 3 to 4 μL/minute. Normal IOP measurements in the human eye, according to widely acceptable consensus, range between 10 and 21 mm of mercury, with an average of 16. The Goldmann equation is a simplified hydraulic algorithm that views aqueous flow as passive non-energy-dependent bulk fluid movement down a pressure gradient (between intraocular pressure [IOP] and episcleral venous pressure [EPV]), with aqueous leaving the eye through the trabecular route. At normal conditions inflow of aqueous equal outflow and the Goldmann equation becomes:

Aqueous Flow=(Trabecular Outflow Facility)×([IOP]−[EVPD])

To maintain an intraocular pressure of 12 to 19 mm Hg (*IOP human eye=15.6±3.2 mm Hg) against a episcleral venous pressure of 7 to 11 mm Hg (**EVP human eye=7.6 to 11.6 mm Hg), the outflow tissues have to provide the necessary resistance. (*Armaly M F. Arch Ophthalmol. 1965; 73:318) (**Glaucoma. In Basic and Clinical Science Course Section 10; American Academy of Ophthalmology, 2007-2008 ed.) Any part of the trabecular outflow pathway, consisting of the trabecular meshwork, the pericanalicular connective tissue, the endothelial lining of Schlemm's canal, Schlemm's canal itself, the collecting channels and aqueous veins, may virtually contribute to the resistance to aqueous outflow. According to the well evidenced morphological features of the uveal and corneoscleral trabecular meshwork, the theoretical resistance to aqueous outflow in this region is negligible. (McEwen W K., 1958. Arch Ophthalmol. 1958; 60:290) (Grant W M., 1963. Arch Ophthalmol. 1963; 69:783-801). The evidenced diameters of the collector channels and aqueous veins are in the scale of tens of μm and theoretical calculations have shown that the contribution of these vessels to the aqueous outflow resistance is negligible. (Rosenquist Ret al. Curr Eye Res. 1989; 8:1233-1240). In the primate eye, little pressure difference exists between the Schlemm's canal pressure and the episcleral veins pressure. (Maepea O, Bill A. Exp Eye Res. 1989 49:645-663) Pericanalicular connective tissue is a distinct layer that surrounds the Schlemm's canal, through which the aqueous flows before reaching the endothelial layer of the inner wall of Schlemm's canal. The above observations suggest that the significant contribution to outflow resistance may stem in the barriers of the schlemm's canal itself. The endothelial cells of Schlemm's canal form a continuous monolayer that run mostly in an equatorial direction. This endothelial lining, similarly to lymphatic vessels, is perfused from outward to inward and the pressure gradient wave moves from the basement membrane to the basal side of the endothelial cell. In another lymphatic like feature, the endothelial cells of the inner wall of Schlemm's canal also develop cytoplasmic processes that interdigitate with similar processes of underlying cells of a second row underneath the canal endothelium. The endothelial cells of Schlemm's canal are bound together by tight junctions. (Raviola G, Raviola E. Invest Ophthalmol Vis Sci. 1981; 21:52) 041-2048). “glaucoma” in the present invention refers to an eye disorder characterized by increased intraocular pressure, excavation of the optic nerve head and gradual loss of the visual field. An abnormally high intraocular pressure is commonly known to be detrimental to the eye, and there are clear indications that, in glaucoma patients, this probably is the most important factor causing degenerative changes in the retina. The pathophysiological mechanism of open angle glaucoma is, however, still unknown. In one particular form of glaucoma, low tension glaucoma, damage may occur at intraocular pressure levels otherwise regarded as physiologically normal. The reason for this could be that the eye in these individuals is unusually sensitive to pressure. “ocular hypertension” in the present invention refers to clinical situation in individuals with an abnormally high intraocular pressure without any manifest defects in the visual field or optic nerve head. Such conditions are usually referred to as ocular hypertension. Individuals with ocular hypertension carry the risk of conversion to glaucoma with the risk being correlated to higher intraocular pressure measurements. Therefore, offering treatment to lower intraocular pressure to subjects with ocular hypertension to prevent conversion to glaucoma is the subject of ongoing debate on the timing of such intervention. The death of retinal ganglion cells is the hallmark of glaucoma, and the cascade of events that lead to cell death include the vrelease of excitatory neurotoxins. “Neuroprotection” in current inventions relate to therapeutic interventions to prevent the death of retinal ganglion cells, which is the hallmark of glaucoma progression. Retinal ganglion cell death, like other neurodegenerative processes, is amenable to neuroprotective interventions. Neuroprotective agents should have the ability to prevent or arrest the cascade of events that lead to cell death. At a preclinical setting, successful neuroprotective agent should allow the neutralization of excitatory neurotoxins. Neroprotection may also prevent “retinal ganglion cell pressure-induced death”. Reported evidence that suggest that elevated intraocular pressure could be a direct trigger of retinal ganglion cell death. (Sappington R M, Chan M, Calkins D J. Invest Ophthalmol Vis Sci. 2006; 47 (7):2932-42) (Agar A, Li S, Agarwal N, Coroneo M T, Hill M A. Brain Res. 2006; 1086 (1):191-200) (Agar A, Yip S S, Hill M A, Coroneo M T. J Neurosci Res. 2000; 60 (4):495-503). “therapeutically effective amount” in the present invention refers to an amount of a compound of the present invention that when administered to the subject in need of therapy it will decrease or prevent the increase in intra ocular pressure in such a magnitude that the clinically desired level of intraocular pressure will be reached or maintained. In other words, the term “therapeutically-effective amount” is that amount of the present pharmaceutical composition which produces a desired result or exerts a desired influence on the particular condition being treated. Various concentrations may be used in preparing compositions incorporating the same ingredient to provide for variations in species (animal, human, etc.) body weight, age of the subject to be treated, the severity of the condition, the duration of the treatment and the mode of administration. “pharmaceutically acceptable derivative” in the present invention refers to any non-toxic salt, ester, salt of an ester, or other derivative of a compound of this invention that, upon administration to a recipient, is capable of providing, either directly or indirectly, a compound of this invention or an active metabolite or residue thereof. Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium and potassium), alkaline earth metal (e.g., magnesium), ammonium, and N.sup.+ (C.sub. 1-4 alkyl) .sub.4 salts. “subject in need of such treatment” in the present invention refers to an animal, preferably a mammal, and most preferably a human in need of such treating or preventing, as judged by their clinical assessment.

“increasing the success rate of glaucoma surgical procedures” in the present invention refers to one embodiment of the present invention were the composition of the present invention is administered substantially together with the performance of the following surgical procedures for the treatment of glaucoma. The composition of the present invention will be administered through different routs of drug delivery in therapeutically effective amount to achieve the following: (a) to provide lowering of intraocular pressure as adjuvant to the surgical procedure itself. (b) to prevent spikes of elevated intraocular pressure that is a frequent side effect of the postsurgical period in some surgical procedures. (c) to provide enhanced permeability to the filtering surfaces of the surgical procedures as adjuvant effect and to prevent failed surgery due to failed filtration function of the filtering surfaces after surgery in some cases.

“substantially together” in the present invention refers to the administration of two interventions either simultaneously or within a period of time where the subject would receive the benefit of both separate interventions including taken together (simultaneous) or with a few seconds to at least about 24 hours of one another (not-simultaneous, but co-administered).

“laser iridotomy” in the present invention refers to a surgical incision into the iris, an intraocular tissue that forms a diaphragm that separates the posterior chamber of the eye from the anterior chamber. At normal conditions, aqueous humor flows through the pupil which is the only natural opening through the iris that is otherwise none permeable to aqueous humor flow. When aqueous humor flow through the pupil is compromised there is a need to create an alternative opening through the iris to allow shunting of the aqueous between posterior and anterior ocular chambers to prevent pressure build up in the posterior chamber. “laser iridoplasty” in the present invention refers to a surgical procedure for the treatment of some glaucoma patients aiming at retracting the peripheral iris via the thermal effect of the laser to widen the iridocorneal angle and facilitate aqueous outflow through this angle. “surgical iridectomy” in the present invention refers to surgical removal of part of the iris indicated when aqueous humor flow through the pupil is compromised and there is a need to create an alternative opening through the iris to allow shunting of the aqueous between posterior and anterior ocular chambers to prevent pressure build up in the posterior chamber. “laser trabeculoplasty” and “selective laser trabeculoplasty” in the present invention refers to the use of a very focused beam of light to treat the angle of the anterior chamber of the eye where the aqueous humor flow out of the eye through a sponge-like tissue, the trabecular meshwork, aiming at facilating aqueous outflow through this portion of the eye. The two types of laser trabeculoplasty are Argon laser trabeculoplasty (ALT) which uses Argon laser, and Selective laser trabeculoplasty (SLT) that uses a lower level laser to open the drainage angle of the eye. Before performing this procedure the patient have to receive to his treated eye a topical drug for lowering intraocular pressure or for preventing intraocular pressure spikes following this procedure. Then, a special microscope (slit lamp) and lens (gonio lens) are used to guide the laser beam to the canals (trabecular meshwork) where fluid drains from the eye and the laser bream creates small burns in the trabecular meshwork aiming at separating beams of trabecular meshwork from each other by contraction forces. “filtering surgery” in the present invention refers to the surgical procedure were a very small piece of the wall of the eye (the sclera) is being removed, aiming at allowing the aqueous humor to drain through the hole that is being created that hopefully will result in substantial lowering of the intraocular pressure. The most common type of filtering procedure is called a trabeculectomy. In trabeculectomy, the opening in the sclera takes the form of a hinging flap that is tightened over a hole (in penetrating trabeculectomy) or a very thin residual of the sclera (in none penetrating trabeculectomy).

“fistulizing procedures” in the present invention refers to a general name for all surgeries that create an alternate pathway for aqueous humor outflow.

“needling” in the present invention refers to needle-assisted cystitomy of a failed filtering bleb following trabeculectomy surgery for glaucoma. Filtering bleb is a space created under the conjunctiva of the eye over the site of the trabeculectomy to serve as a receiver reservoir for the aqueous humor that is draining through the trabeculectomy and it serves as a transit station before allowing the aqueous to drain further away from the filtering bleb. The bleb takes the form of bulge in the conjunctival surface. Pathological processes like fibrosis turn successful bleb into a non-functioning bleb that is more like a cyst than a filtering bleb. At this situation a cystitomy is performed with a fine needle, usually combined with subconjunctival injection of 5-fluorouracil as anti-fibrotic agent. The procedure is referred to also as “needling revision” of poorly functioning filtering blebs. “glaucoma drainage device” in the present invention refers to a tube that leads to a reservoir wherein the tube is inserted into the anterior chamber of the eye without touching the cornea while the reservoir is implanted subconjunctively. several polymers with different properties have been utilized to manufacture the tube and the receiving reservoir.

The reservoir is semi permeable to allow the aqueous to drain further away and may be with a valve to control the aqueous drainage. “cyclodiathermy” in the present invention refers to the destruction of a portion of the ciliary body by diathermy aiming at decreasing the production rate of aqueous humor by the ciliary body and eventually decreasing intraocular pressure.

“cyclocryotherapy” in the present invention refers to the destruction of a portion of the ciliary body by cryotherapy aiming at decreasing the production rate of aqueous humor by the ciliary body and eventually decreasing intraocular pressure. “ocular” in the present invention refers to administering drug directly to the eye. “periocular” in the present invention refers to administrating drugs to the orbital space that surrounds the eye globe. “orbital” in the present invention refers to drug administration to the relatively deeper orbital tissues. “topical” in the present invention refers to administering drugs to the surface of the eye. This goal is achieved by the application to the eye surface one or more of the following: “eye-drops”, “sprayed formulations”, “suspensions”, “ointments”, “gels”, “hydrogels”, “viscosified solution”, “formulation loaded in contact lens”, “formulation loaded in collagen shield”, and “formulation loaded in ocular insert”. (see: Abdulrazik M, Behar-Cohen F, Benita S. Drug Delivery Systems for Enhanced Ocular Absorption. In Enhancement in Drug Delivery, Touitou E, Barry B W, Eds., Taylor and Francis, pp. 489-526, 2006). “corneal” and “intra-corneal” in the present invention refers to administering a drug to the cornea while taking necessary measures to minimize drug penetration through other ocular tissues. In preferred embodiment of the present invention the composition of the invention is administered in repeated small volume applications (of few micro-liters each) on the corneal surface to allow penetration through the cornea while minimizing penetration through the conjunctival surface, aiming at allowing drug accumulation in the anterior chamber and eventually the drainage of the dug to the assumed site of action, the schlemm's canal. The same goal is achieved by similar application of the composition of the present invention on the upper third of the cornea following partial and gentle debridement of the surface epithelium of the cornea in this region of the corneal surface. The same goal is achieved again by using penetration enhancement agents, like benzalkonium chloride, selectively by applying them just on part of the cornea were the composition of the present invention will be applied soon after then or substantially together. In yet another embodiment of the present invention, composition of the invention is implanted or injected to the stroma of the cornea (upper third), through the cornea into the anterior chamber of the eye, or to the scleral tissue adjacent to the schlemm's canal. “Subconjunctival” in the present invention refers to administering drug behind the conjunctiva. In this case the drug penetration is not challenged by the surface barriers of the conjunctiva. In a preferred embodiment of the present invention the active composition could be injected or implanted supconjuctively at site adjacent to the schlemm's canal. “subtenon” in the present invention refers to drug administration directly to the subtenon space by injecting, pumping, or implanting drugs or drug delivery systems directly to the subtenon space or by allowing intimate contact of these systems with the tenon's fascia.

Tenon's fascia is a tissue that takes the form of a coating that surrounds the globe and extraocular muscles and is situated under the conjunctiva.

“episcleral” in the present invention refers to drug administration directly to the episcleral tissue by injecting, pumping, or implanting drugs or drug delivery systems directly to the episclera tissue or allowing intimate contact of these systems with the episclera. The episclera is a fine vascularized tissue the form an external lining of the sclera. “intra-scleral” in the present invention refers to to drug administration directly to the sclera tissue by injecting, pumping, or implanting drugs or drug delivery systems directly to the sclera tissue or allowing intimate contact of these systems with the sclera. “Intracameral” in the present invention refers to drug administration into the anterior chamber of the eye. The anterior chamber is the most anterior compartment of the eye filled with aqueous humor, that flows in from the posterior chamber of the eye were it is being secreted by the ciliary body, with the posterior surface of the cornea as its anterior border and the anterior surface of the iris as its posterior border. The intracameral drug administration could be accomplished by injection, pumping, or by implants. “Intravitreal” in the present invention refers to administering drugs into the vitreous cavity. The vitreous cavity is the space that consists most of the volume of the core of the eye with the lens and its suspension system (the zonules) as its anterior border and the retina and its coating as the peripheral border. The intravitreal drug administration could be accomplished by injection, pumping, or by implants. “peribulbar or retrobulbar injections or implants” in the present invention refers to drug administration to the space that surrounds the eye, the orbital space. Priorbital injections are injections to the orbital space that surrounds the eye and usually means the more anterior orbital space, while retrobulbar injections is performed with long-enough needle to reach the deep orbital space. In both cases, the orbital space serves as drug reservoir for later drug permeation towards the ocular tissues.

“glaucoma filtering bleb” in the present invention refers to the space created under the conjunctiva of the eye over the site of the trabeculectomy to serve as a receiver reservoir for the aqueous humor that is draining through the trabeculectomy and it serves as a transit station before allowing the aqueous to drain further away from the filtering bleb. The bleb takes the form of bulge in the conjunctival surface.

“sclerotomy pocket of trabeculectomy” in the present invention refers to the space under the hinging sclera flap in trabeculectomy surgery for glaucoma. In trabeculectomy, the opening in the sclera takes the form of a hinging flap that is tightened over a hole (in penetrating trabeculectomy) or a very thin residual of the sclera (in none penetrating trabeculectomy).

In one embodiment of the present invention the composition of the invention is applied by injection, pumping, or implantation to the filtering bleb or the sclerotomy pocket of trabeculectomy to achieve (i) permeability enhancement of the filtering surfaces and (ii) sustained lowering of intraocular pressure.

“administration is on an as needed basis” in the present invention refers to a preferred embodiment wherein the composition of the current invention is administered on an as needed basis, well known to those skilled in the art as pro re nata dosing.

### Insulin

Insulin is composed of two, alpha and beta, peptide chains with several inter and intra chain disulphide bonds. The three-dimensional structure of the insulin molecule (insulin monomer), essentially the same in solution and in solid phase, exists in two main conformations. These differ in the extent of helix in the B chain which is governed by the presence of phenol or its derivatives. In acid and neutral solutions, in concentrations relevant for pharmaceutical formulation, the insulin monomer assembles to dimers and at neutral pH, in the presence of zinc ions, further to hexamers. Many crystalline modifications of insulin have been identified but only those with the hexamer as the basic unit are utilized in preparations for therapy. The insulin hexamer forms a relatively stable unit but some flexibility remains within the individual molecules. The intrinsic flexibility at the ends of the B chain plays an important role in governing the physical and chemical stability of insulin. Insulin may undergo structural transformations resulting in aggregation and formation of insoluble insulin fibrils. Physical modifications of the secondary to quaternary structures (denaturation, aggregation, and precipitation) are known to affect VEGFR-1 agonists, SPARC antagonists, insulin and insulin preparations during storage and use. A variety of chemical changes of the primary structure have been suggested to produce insulin derivatives with favorable stability. Although the exact mechanism of fibril formation is still obscure, it is now clear that the initial step is an exposure of certain hydrophobic residues, normally buried in the three-dimensional structure, to the surface of the insulin monomer. Therefore, most methods stabilizing insulin against fibrillation share the property of being able to counteract associated insulin from being disassembled. Chemical deterioration of insulin during storage of pharmaceutical preparations is mainly due to two categories of chemical reactions, intermolecular transformation reactions and hydrolysis.

Reportedly, a predominant hydrolysis reaction is a hydrolytic cleavage of the backbone A chain, presumably autocatalyzed by an adjacent insulin molecule, has been identified in insulin preparations containing rhombohedral crystals in combination with free zinc ions. (Brange J, Langkjoer L. Pharm Biotechnol. 1993; 5:315-50)

“Placenta growth factor” (PlGF) was discovered in human placenta in 1991, about 2 years after the identification of vascular endothelial growth factor (VEGF), and was so named because it is abundant in placental tissue (Maglione et al., Proc Natl Acad Sci USA, 1991; 88:9271-6). This dimeric glycoprotein, with 3 identified isomers which differ in size and binding properties (PlGF-1 (PlGF131), PlGF-2 (PlGF152) and PlGF-3 (PlGF203)), was later identified as a member of the VEGF family as the molecule shares about half of a homologous domain with the platelet derived growth factor-like region of VEGF (Cao et al., Biochem Biophys Res Commun, 1997; 235 (3):493-8). PlGF homodimers exclusively bind with high affinity to the tyrosine kinase VEGF receptor-1 (Flt-1), but have little effect on angiogenesis in vitro. Indeed, PlGF had a negligible role in physiological angiogenesis and vascular development but do play a role in pathological angiogenesis. PlGF and VEGFR-1 are minimally expressed in adult quiescent vasculature, but are markedly upregulated during pathological conditions (Carmeliet et al., Nat Med. 2001; 7 (5):575-83). Shih et al have demonstrated that PlGF-1 intervention didn't carry the risk of provoking retinal neovascularization (Shih et al., J Clin Invest. 2003; 112 (1):50-7). “Insulin-Like Growth Factor” (IGF) is a growth factor structurally related to insulin. IGF-1 is a single chain peptide with three intermolecular disulfide bridges. IGF-I is the primary protein involved in responses of cells to growth hormone (GH). IGF-I is produced in response to GH and then induces subsequent cellular activities, particularly on bone growth. Subsequent studies have demonstrated, however, that IGF-1 has autocrine and paracrine activities in addition to the initially observed endocrine activities on bone. The IGF-1 receptor, like the insulin receptor, has intrinsic tyrosine kinase activity. Owing to their structural similarities IGF-1 can bind to the insulin receptor but does so at a much lower affinity than doe's insulin itself.

Insulin, PlGF, and/or IGF may undergo structural transformations resulting in aggregation and formation of insoluble Insulin, PlGF, and/or IGF fibrils. Physical modifications of the secondary to quaternary structures (denaturation, aggregation, and precipitation) are known to affect Insulin, PlGF, and/or IGF and their preparations during storage and use. A variety of chemical changes of the primary structure have been suggested to produce Insulin, PlGF, and/or IGF derivatives with favorable stability. Although the exact mechanism of fibril formation is still obscure, it is now clear that the initial step is an exposure of certain hydrophobic residues, normally buried in the three-dimensional structure, to the surface of the PlGF-1 and/or IGF-1 monomer. Therefore, most methods stabilizing PlGF-1 and/or IGF-1 against fibrillation share the property of being able to counteract associated PlGF-1 and/or IGF-1 from being disassembled. Chemical deterioration of PlGF-1 and/or IGF-1 during storage of pharmaceutical preparations is mainly due to two categories of chemical reactions, intermolecular transformation reactions and hydrolysis.

As the skilled artisan will appreciate, several strategies may be used to enhance purity, stability, solubility, activity, or target selectivity, as well as to reduce or mute immunogenicity of a polypeptide. Such methods are well known in the art and have been previously described, as highlighted below. Among them, recombinant DNA technology, well known to those skilled in the art, can be used to create novel mutant proteins or muteins including single or multiple amino acid substitutions, deletions, additions or fusion proteins. Therefore, the Insulin, PlGF, and/or IGF to be used to achieve the subject matter of the present invention could be native (e.g., wild-type, naturally occurring, or allelic variant) Insulin, PlGF, and/or IGF, synthetic Insulin, PlGF, and/or IGF, semisynthetic Insulin, PlGF, and/or IGF. active dimeric form of Insulin, PlGF, and/or IGF, active multimeric form of Insulin, PlGF, and/or IGF, Insulin, PlGF, and/or IGF isoforms and isomers, Insulin, PlGF, and/or IGF with modified stereochemistry. Likewise, strategies that utilize analogs of Insulin, PlGF, and/or IGF, peptide mimetic of Insulin, PlGF, and/or IGF, biologically active fragment of Insulin, PlGF, and/or IGF, Insulin, PlGF, and/or IGF that underwent amino acid substitutions, and Insulin, PlGF, and/or IGF that underwent amino acid insertions, are widely available to the skilled artisan and can allow the fine-tuning of target selectivity to reach highest activity and stability with minimal side-effect rates. Many other methods to reach the same goal are also well known and will be apparent to the ordinarily skilled artisan. Therefore, insulin, PlGF, and/or IGF that was subject to other gene or protein engineering modifications and chemically modified derivatives or salts of insulin, PlGF, and/or IGF can be utilized to enhance its purity, stability, solubility, activity, and target specificity. See also: (Cleland J L and Craik C S eds., Protein engineering: principles and practice, Wiley-liss, 1996), (Alberghina L, ed., Protein engineering for industrial biotechnology, CRS Press, 2000), (Budisa H, ed., Engineering the genetic code, Wiley-VCH Verlag, Weinheim, 2006), (Niazi S K, ed., Handbook of biogeneric therapeutic proteins, CRS Press, Taylor & Francis, Boca Raton, 2006), (Nail S L and Akers M J, eds., Development and manufacture of protein pharmaceuticals, Kluwer Academic/Plenum Publishers, New York, 2002).

Frequency of PlGF-1 and/or IGF-1 Therapy

As the skilled artisan will appreciate, different dosage and treatment regimens of insulin, PlGF, and/or IGF may be needed to achieve the subject matter of the present invention for any particular subject in need of therapy, depending upon a variety of factors, including type, severity and course of the Glaucoma or ocular hypertension being treated, as well as the bioavailability characteristics of insulin, PlGF, and/or IGF preparation administered, concurrent treatment with other co-administered therapeutic agents, and other relevant circumstances. Insulin, PlGF, and/or IGF could be administered once or at multiple times. Multiple administrations could be performed at intervals ranging from ONCE-PER-MINUTE, THROUGH hourly, And could be daily, weekly, biweekly, monthly, biyearly, yearly. However, intervals can also be more or less frequent than the above. Insulin, PlGF, and/or IGF could be administered independently or co-administered with one or more additional therapeutic agent disclosed herein.

### Combination Therapy

insulin, PlGF, and/or IGF could be conjugated to another drug, transport vector, or permeability enhancing peptide by avidin-biotin technology, polyethylene glycol linkers or other chemical linkers.

Combination therapies according to this invention exert a synergistic effect. The use of such combinations is also advantageous to reduce the dosage of a given conventional therapeutic agent that would be required for a desired therapeutic or prophylactic effect. Combinations treatments may also reduce or eliminate the side effects of conventional single agent therapies.

Combinations may also increase the efficacy of a conventional agent without increasing the associated toxicity. In one embodiment of the present invention, a combination therapy is consisting of insulin, PlGF, and/or IGF co-administered or conjugated to at least one conventional antiglaucoma drug selected from the group consisting of a prostaglandin analogues, beta-blockers, alpha2-adrenergic agonists, carbonic anhydrase inhibitors, miotics and nonselective adrenergic agonists (epinephrine and its derivatives). (See also: Physicians' Desk Reference (PDR) for Ophthalmic medicines, 2006)

In another embodiment of the present invention, a combination therapy is consisting of insulin, PlGF, and/or IGF co-administered or conjugated to at least one compound that possess antiglaucoma or anti ocular hypertensive activity, selected from the group consisting of calcium channel antagonists, preferably verapamil and nifedipine, potassium channel antagonists, nitrovasodilators, nitroglycerin, isosorbide dinitrate, nitroprusside, minoxidil, atrial natriuretic factor, phenylimino-imidazoles, serotonergic receptor antagonists, preferably ketanserin, angiotensin converting enzyme (ACE) inhibitors, H1-antihistamines, preferably antazoline and pyrilamine, delta 9-tetrahydrocannabinol, cardiac glycosides, preferably ouabain and digoxin), A3 subtype adenosine receptor antagonists, a cAMP modulator, cyclic GMP, preferably 8-Bromo cyclic GMP, cyclic guanosine 3′,5′-monophosphate specific phosphodiesterase type 5 (cGMP-PDE5) inhibitor, hyaluronidase, proteases, matrix metalloproteinases, transforming growth factor-beta, cytochalasin B, cytochalasin D, ethacrynic acid, tienilic acid, aminotetralins, staurosporine, oncomodulin, latrunculins, alkaloids, antiestrogens, ethyleneglycol bis (aminoethylether) tetraacetate (EGTA) and ethylenediamine tetraacetic acid (EDTA).

Corticosteroids are being used locally or systemically for a wide range of indications in clinical ophthalmology. One particular complication with this therapeutic regimen is steroid induced glaucoma. Steroid induced glaucoma is a form of open angle glaucoma that usually is associated with topical steroid use, but has been reported also following inhaled, oral, intravenous, periocular, or intravitreal steroid preparations. The incidence of this complication varies, being 6-30% among the normal population using topical steroids, but this estimates was reportedly more than doubled in cases with family history of glaucoma. Drug induced glaucoma is similar to steroid induced glaucoma but this name is used when the causative drug is other than a corticosteroid. In one embodiment of the present invention, insulin, PlGF, and/or IGF, alone or in one of the aforementioned combinations, is coadministered or conjugated to a corticosteroid.

In another embodiment of the present invention, insulin, PlGF, and/or IGF, alone or in one of the aforementioned combinations, is coadministered or conjugated to a drug that can cause drug-induced glaucoma. Neovascular glaucoma is a term used to describe secondary glaucoma that develops following the growth of neovascular tissue at the sites of aqueous humor outflow. This condition is usually secondary to ischemic disorders like diabetic retinopathy, central retinal vein occlusion, severe carotid artery disease, severe ischemic retinopathies due to sickle cell disease, and central retinal artery occlusion. In one embodiment of the present invention, insulin, PlGF, and/or IGF-1, alone or in one of the aforementioned combinations, is coadministered with or conjugated to an antiangiogenic compound as a combination therapy to treat or prevent neovascular glaucoma. In another embodiment of the present invention, the combination therapy that comprises insulin, PlGF, and/or IGF, alone or in one of the aforementioned combinations, together with a antiangiogenic agent is meant to prevent a possible neovascularization side effect of the insulin, PlGF, and IGF intervention itself. Polypeptides are hydrophilic macromolecules that are virtually impermeable through ocular barriers that are characterized by lipophilic cell membranes and intercellular pores that are too small to allow the permeation of most polypeptides. “Permeability enhancing agents” are compounds that, when coadministered with or conjugated to ocular hypotensive peptides of the present invention produces an increase in transport of this polypeptide across ocular barriers. In one embodiment of the present invention, the permeability enhancing agent is selected from the group consisting of bile salt, surfactant, chelating agent, phospholipid additive, medium-chain fatty acid, hydrophobic penetration enhancer, long-chain amphipathic molecule, cyclodextrin or beta-cyclodextrin derivative, N-acetylamino acid or salt, glycerol ester of acetoacetic acid, salicylic acid derivative, and enamine.

In another embodiment of the present invention, ocular hypotensive peptides of the present invention is co-administered with or conjugated to a permeability enhancing agent. In a preferred embodiment, the permeability enhancing agent is a permeability enhancing peptide, preferably transferrin.

### Formulations

Pharmaceutically acceptable composition is formed by incorporating the ocular hypotensive peptides of the present invention, with the desired degree of purity, in a nontoxic formulation using one or more suitable and pharmaceutically acceptable carriers, excipients, dilutents, and stabilizers. Pharmaceutical compositions can be formulated by standard techniques widely available to the ordinarily skilled artisan.

See also: (United States Pharmacopeia/National Formulatory (USP NF), 2007), (Rowe R C, et al., Handbook of Pharmaceutical Excipients, 5th ed., 2006), (Martin's Physical Pharmacy and Pharmaceutical Sciences, Lippincott Williams & Wilkins, 2005), (Remington the science and practice of pharmacy, 21 Ed., Lippincott Williams & Wilkins, 2005)

### Insulin PlGF, and IGF-1 Stabilization:

Rational strategies can be devised for stabilization of the ocular ocular hypotensive peptides of the present invention. Several strategies for protein stabilization are widely available to the ordinary skilled artisan, depending on the mechanism involved. For example, active insulin, PlGF and IGF conformations could be stabilized by introducing a disulfide bond between chains of a polypeptide or between two polypeptides. One strategy to achieve this goal is by introducing point mutations to cystine in the relevant polypeptide chains (See, for example: Reiter Y. et al. Nat. Biotech.; vol. 14: pp. 1239-1245, 1996). On the contrast, if for instance the mechanism of polypeptide aggregation is discovered to be intermolecular S—S bond formation, then stabilization could be achieved by modifying sulfhydryl residues to eliminate such tendency.

Example of protein stabilizing strategies in a solid formulation is the water content control. Water content could be controlled to keep the water activity in the formulation at optimal levels that prevent polypeptide destabilization interactions. In another example of protein stabilizing strategies, cross-linking methodologies could be used to stabilize a desired active conformation.

Examples of cross-linking methodologies include UV-cross-linking, and treatment of protein with formamide or glutaraldehyde. Further example of protein stabilizing strategies in a solid formulation is the use of sucrose and sucrose based polymers, to provide significant protection against peptide or protein aggregation during solid-phase incubation and to enhance the stability of solid proteins embedded within polymer matrices. Compounds such as gelatin and collagen can also serve as stabilizing or bulking agents to reduce denaturation and aggregation of proteins.

Certain solubilization agents could be also serve as stabilizing agents that significantly inhibits protein aggregation, such as cyclodextrins, cyclodextrin dimers, trimers and tetramers, which selectively bind hydrophobic side chains of polypeptides.

Example of protein stabilizing strategies in solution formulations is the use of various additives such as polyols, amino acids, collagen, gelatin, and various salts. Several other compounds have shown the ability to reduce the aggregation of polypeptides and proteins. Examples of such compounds are dextrans, diethylaminoethyl dextran, carboxymethyl cellulose, glycopeptides, polyamino acids, and polyethylene glycol.

In another approach, the stability of protein is enhanced by the inhibition of enzymatic degradation processes. Reportedly, some bile salts and fusidic acid derivatives are capable of inhibition proteolytic degradation of proteins. Since divalent cations are co-factors for many proteases, some chelating agents, such as EDTA, can be used in suitable concentrations that ensure efficient inhibition of relevant proteases. Besides EDTA and similar chelating agents, some mucoadhesive polymers have also the capability of divalent cation complexation.

Examples of such mucoadhesive polymers are polyacrylates and polycarbophil. Boro-amino acids, which are produced by incorporating the boron atom, can form a tetrahedral boronate ion and could be a mimetic of peptides in their transition state and serve as amino-peptidases substrate. Therefore boro-amino acid could provide peptide sparing effect in tissues rich of amino-peptidases hydrolysis activity by acting as reversible inhibitors of such enzymes. Several other peptide mimetics could be used selectively to block the interaction between active peptides and degradation enzymes. The development of complexes of anti-enzymatic agents with selected polymers such as Chitosan-EDTA, allow the incorporation of such complexes in drug delivery systems that serve as carriers of ocular hypotensive peptides of the present invention and provide surface protective layer against enzymatic degradation of those peptides.

### Salts

The formation of salts of a composition is way of modulating its physicochemical properties to allow for favorable drug disposition. Pharmaceutically acceptable salt should be non-toxic and should not alter the biological activity of the active composition. A selected pharmaceutically acceptable salt of a peptide could be prepared by widely available to the ordinary artisan.

Examples include the treatment of the free base with an inorganic or organic acid. Inorganic acids could be selected from group consisting of hydrobromic acid, hydrochloric acid, nitric acid, phosphoric acid, sulfamic acid, sulfonic acid, sulfuric acid, and mixtures thereof. Organic acids could be selected from group consisting of acetic acid, alpha-hydroxy acid, aspartic acid, benzenesulfonic acid, benzoic acid, cinnamic acid, citric acid, cyclohexylsulfamic acid, cyclohexylsulfonic acid, ethanesulfonic acid, fumaric acid, galacturonic acid, glucuronic acid, glutamic acid, glycolic acid, lactic acid, maleic acid, malonic acid, mandelic acid, methanesulfonic acid, oxalic acid, p-toluenesulfonic acid, pyranosidyl acid, pyruvic acid, quinic acid, salicylic acid, succinic acid, sulfonic acid, tartaric acid, or mixtures thereof.

Examples of pharmaceutically acceptable salts of a peptide include acetate, acrylate, benzenesulfonate, benzoate, bicarbonate, bisulfate, bisulfite, bitartrate, borate, bromide, butyne-1,4-dioate, calcium edetate, camsylate, carbonate, chloride, caproate, caprylate, clavulanate, chlorobenzoate, citrate, cyclohexylsulfamate, cycloexylsulfonate, decanoate, dihydrochloride, dihydrogenphosphate, dinitrobenzoate, diphosphate, edetate, edislyate, estolate, esylate, ethanesulfonate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, hydroxybenzoate, hydroxybutyrate, iodide, isobutyrate, isothionate, lactate, lactobionate, laurate, malate, maleate, malonate, mandelate, mesylate, metaphosphate, methanesulfonate, methoxybenzoate, methylbenzoate, methylsulfate, monohydrogenphosphate, mucate, napsylate, naphthalene-1-sulfonate, naphthalene-2-sulfonate, nitrate, oleate, oxalate, pamoate, palmitate, pantothenate, phenylacetates, phenylbutyrate, phenylpropionate, phthalate, phosphate, polygalacturonate, propanesulfonate, propionate, propiolate, p-toluenesulfonate, pyrophosphate, pyrosulfate, quinate, salicylate, stearate, subacetate, suberate, succinate, sulfate, sulfonate, sulfite, tannate, tartrate, teoclate, tosylate, triethiodode, and valerate salts.

### Excipients

Additives to ophthalmic formulations of bioactive peptides optionally include preservatives, buffers, tonicity agents, antioxidants, stabilizers, wetting and clarifying agents, viscosity increasing agents, and antibacterial, antiviral, or antifungal agents.

Examples of preservatives include benzalkonium chloride, benzethonium, phenylethyl alcohol, chlorobutanol, thimerosal and the like. Examples of buffers include boric acid, sodium and potassium bicarbonate, sodium and potassium borates, sodium and potassium carbonate, sodium acetate, sodium biphosphate, Tris, and the like. Examples of tonicity agents are dextran, dextrose, glycerin, potassium chloride, propylene glycol, sodium chloride and the like. Examples of antioxidants and include sodium and potassium bisulfate, sodium and potassium metabisulfite, sodium thiosulfate, thiourea and the like. Examples of stabilizers include EDTA, EGTA, DTPA, DOTA, ethylene diamine, bipyridine, 1,10-phenanthrolene, crown ethers, aza crown, catechols, dimercaprol, D-penicillamine and deferoxamine. Examples of wetting and clarifying agents include polysorbate 80, polysorbate 20, poloxamer 282 and tyloxapol. Examples of viscosity increasing agents include dextran, gelatin, glycerin, hydroxyethyl cellulose, hydroxypropylmethyl cellulose, lanolin, methylcellulose, petrolatum, polyethylene glycol, polyvinyl alcohol, polyvinyl polyvinylpyrrolidone, carboxymethyl cellulose and the like.

Necessary antibacterial, antiviral, or antifungal agents are added in sufficient amounts to the above excipients according to guidelines well known to the skilled artisan.

Aqueous formulations are based on water, buffered water, saline, glycine, hyaluronic acid and the like. Aqueous formulations may be used as is, or lyophilized. Lyophilized preparation is being revived prior to administration by adding sterile solution.

Ointment formulations are based on ingredients such as petrolatum, paraffin, oleic acid, mineral oil, lanolin, glycerol, polysorbates, glycol ethers, polyethylene glycols and waxes.

Aerosol formulations have usually three main components. Beside active compound and propellant, the aerosol formulations are based on esters, such as glycerides, of fatty acids, such as caproic, octanoic, lauric, palmitic, stearic, linoleic, linolenic, olesteric and oleic acids. Surfactants and cosolvents are usually needed to prevent unwanted aggregation of the active compound.

### Solid Formulations

Solid formulations usually utilize pharmaceutical grades of cellulose, lactose, polysaccharide, starch, sucrose, and the like. Examples of polysaccharides useful for solid formulations are alginate, carboxymethyl cellulose, carboxymethyl chitosan, carrageenan, crosscarmellose, curdlan, gellan, gum Arabic, hydroxypropyl carboxymethyl cellulose, karaya gum, mesquite gum, pectin, poly acrylic acid, xanthan, and the like. Methods described herein can be generally adapted for use with such ingredients. Example of strategies for the modulation of the physicochemical properties of polysaccharides is the acylation of gellans. Acylated gellan tends to form soft, elastic, transparent and flexible gels. On the contrast, de-acylated gellans are hard, relatively non-elastic brittle gels. Active compound in a solid formulation can be attached to a backbone either directly or by a linker arm. The backbone can be composed of monomer units that are covalently or non-covalently linked. Examples of covalent backbones include oligosaccharides, peptides, lipids and other cross-linked monomers. Poly lactic acid (PLA) and poly glycolic acid (PGA) and their copolymer (PLGA) are among the most widely used biodegradable polymers. They are approved for human use by worldwide health authorities and are degraded into nontoxic compounds (lactic acid and glycolic acid, respectively) following hydrolysis and enzymatic cleavage. These polymers undergo bulk erosion and drug diffusion may change according to the erosion rate of the polymer matrix. Biodegradable polymeric systems were used as colloidal systems and inserts, and also as an ophthalmic drug delivery system (DDS) for subconjunctival, scleral, intracameral, or intravitreal application.

Nonbiodegradable polymers allow the design of long term sustained release DDS but carry the disadvantage of subsequent intervention to remove the DDS at the end of its functioning life.

In some embodiment of the present invention, the active compound can be incorporated in collagen shields or contact lenses that can serve as time release DDS on the ocular surface.

Collagen shields are manufactured from porcine scleral tissue and commercially available (bio-Cor, Bauch and Lomb) with three dissolution times of 12, 24, and 72 hours, depending on the level of collagen crosslinking induced during the manufacture process. Hydrophilic drugs are entrapped within the collagen matrix when the dry shield is soaked in aqueous solution of the drug while water insoluble drugs are incorporated into the shield during the manufacturing process. The commercially available shields consist of a contact-lens-shaped dry product which can absorb fluid up to 60% water content when soaked in aqueous solution, and is biodegradable in a time frame according to the programmed dissolution time. Therapeutic contact lenses can act as a pre-corneal drug reservoir allowing for higher drug concentrations to be in contact with ocular surface.

(See also: Abdulrazik M, Behar-Cohen F, Benita S. Drug Delivery Systems for Enhanced Ocular Absorption. In Enhancement in Drug Delivery, Touitou E, Barry B W, Eds., Taylor and Francis, pp. 489-526, 2006)

### Liposomes

Liposomes are vesicles consisting of an aqueous compartment core surrounded by a lipid bilayer that mimics a cell membrane. The aqueous core can be surrounded by either a single lipidic bilayer (unilamellar liposomes) or concentric multiple bilayers (multilamellar liposomes).

Liposomes with multiple bilayers are termed ‘multilamellar vesicles’ (MLV), whereas the unilamellar liposomes are termed ‘small unilamellar vesicles’ (SUV) or ‘large unilamellar vesicles’ (LUV) according to their size (less or more than 100 nm accordingly). The bilayer of the liposomes is formed from a double array of phospholipids with their lipophilic tails facing each other and their hydrophilic heads embedded either in the aqueous core or in the aqueous solution that separate vesicles from each other. The typical liposome bilayer components are phophatidylecholine (PC) as the main backbone and a small amount of cholesterol (Chol) to improve the stability of the bilayer array. However, other amphiphiles such as negatively charged phospholipids (phosphatidylethanolamine (PE), phosphatidylserine, etc.) or cationic lipids (stearylamine, DOTAP, etc.) can be used to achieve the desired electrostatic bilayer properties. The incorporation of phospholipids with polyethyleneglycol (PEG) moiety on the hydrophilic head increases the stability of the liposomal vesicle and decreases the rate of uptake by the reticuloendothelial system (RES), probably by steric interference with the foreign particle elimination mechanisms of the RES. These “PEGylated” liposomes have shown excellent prolonged circulation in the serum and are termed “stealth liposomes”. One example of “PEGylated” liposome bilayer molecular composition consists of 10:1:0.5 of PC:Chol:PEG-PE.

However, bilayer components nature and proportions are usually tailored to meet the optimal conditions for a specific incorporated drug. Liposomes are usually prepared by the reverse phase evaporation method or its modification, consisting of vigorous mechanical mixing of the water phase and low boiling-point organic solvents that contain the phospholipids and other bilayer components. Liposomes can incorporate a wide variety of molecules and peptides. Hydrophilic compounds should be incorporated in the aqueous phase while lipophilic compounds are added to the bilayer components. Because the lipid bilayer occupies only a small volume, the incorporating capacity of liposomes for lipophilic drugs is far more limited than for hydrophilic drugs. However, pro-drug strategy can allow the incorporation of lipophilic drugs in the aqueous phase and vice versa.

### Micelles

Amphiphilic molecules (surfactants) can assemble into nanoscopic supramolecular structures with a hydrophobic core and a hydrophilic shell micellar arrangement. As surfactant concentration is increased in aqueous solutions, the separated molecules aggregate into micelles upon reaching a concentration interval known as the critical micellar concentration (CMC).

In micellar systems, nonpolar molecules are solubilized within the internal micelle hydrophobic core, polar molecules are adsorbed on the micelle surface and substances with intermediate polarity are distributed along surfactant molecules in intermediate positions.

Micellar ocular drug delivery systems are based on non-toxic and non-irritant materials and are stable enough to achieve a reasonable shelf life. The non-ionic triblock copolymers, like poly ethylene oxide-poly propylene oxide-polyethylene oxide (PEO-PPO-PEO), are among the most widely used polymeric micelles.

### Emulsions

Oil in water emulsions are two-phase systems formed by the dispersion of oil (the internal phase) in water (the external phase) and stabilized by at least one surfactant. Submicron emulsion (SME), and nanoemulsion are interchangeable terms used to describe a kinetically stable emulsion system characterized by a droplet size in the nano-range. Microemulsion is a term used to emphasis certain properties of an emulsion. Microemulsions are also a nano-range two-phase systems prepared from oil, water and surfactant, but usually more surfactants are used (up to 10% compared to 1% in simple emulsions). Co-surfactants in microemulsions are required to achieve very low interfacial tensions that allow self emulsification and enhanced thermodynamic stability. The surface charge of the emulsion could be negatively charged, neutral or positively charged.

### Nanocapsules

Nanocapsules are colloid systems with an oil core and biodegradable polymeric coating.

Nanocapsules can be prepared by different approaches like, interfacial precipitation, interfacial polymerization, interfacial deposition, and self assembly procedures. The dispersion stability are mainly determined by the nature of outer coating and the type of the used surfactant.

### Particulate Drug Delivery Systems (Micro/Nano Particles)

In general, particulate drug delivery systems can offer more possibilities for controlled drug release. Furthermore, particulate systems are basically more stable than other colloidal systems, and since they can be freeze-dried for long term storage, extended shelf life is feasible.

Particulate carriers meet the basic needs of advanced ocular drug carriers, being nontoxic, non-immunogenic, biocompatible, uniform and biodegradable in a predictable pace. In addition, they have the ability to provide protection for the delivered molecules while interacting with the ocular tissues. Examples of materials used for the preparation of nanoparticles are biodegradable polymers (PLGA and the like), chitosan, albumin, solid lipid, and gold. Surface modifications of nanoparticles with a sterically stabilizing layer can modulate their in vivo biodistribution and lower their tendency to aggregate with biomolecules. Longer residence times on the ocular surface can be achieved if the nanoparticles are coated with a mucoadhesive or charged polymer.

### Dendrimers

Dendrimers is a name used for polyamidoamine (PAMAM, Starburst™), polypropyleneimine-diaminobutane (DAB), polypropyleneimine- diaminoethane (DAE), polyethyleneoxide-carbosilane (CSi-PEO), polyether, and similar macromolecules with a highly branched globular nanometric structure that comprises branches radiating from a central core. Dendrimers vary in the flexibility of branches and type of peripheral functional groups. Branches can terminate at charged and uncharged amino, carboxyl or hydroxyl groups. Most of the described dendrimers are liquid or semi-solid polymers that are not soluble in aqueous solutions and their in vivo toxicity remains a concern. However, innovative synthetic chemistry has allowed the formation of dendrimers tailored for drug delivery. Dendrimers are classified and designated according to the surface terminating group quality and quantity. For amine and sodium carboxylate terminating groups, the designation is PAMPAM; for amine and carboxylic acid groups, DAB; for amine groups, PEA; for polyethyleneoxide, CSi-PEO; and finally, for carboxylate and malonate groups, ‘Polyether’. Full generations (G1, 2, 3, etc.) describe amine terminating groups (except for ‘Polyether’ Go or G2 where it refers to carboxylate and malonate groups, and for PAMAM G2(OH) and G4(OH) where it refers to hydroxyl group), while the half generations (G1.5, 2.5, 3.5, etc.) describe carboxylic acid or sodium carboxylate terminating groups. The number of surface terminating groups, and consequently the molecular weight of the macromolecule, rises with higher generations. Improved loading capacity of dendrimer can be achieved by the process of ‘activation’, a term used to describe a process comprising a heat treatment in a solvent that manipulates the structure of dendrimers where some branches are trimmed away, while keeping the general topology and size, resulting in more flexible molecules. The reduced structural density in the intramolecular core allows higher levels of drug loading and enhanced penetration capabilities.

“physical penetration enhancers” in the present invention refers to the enhancement of drug penetration through tissue barriers by wide array of interventions that share the common ground of delivering energy that its strong enough to cause a very short and reversible change in the properties and configuration of cell membrane and tissue barriers in a way that will allow co-administered drugs to benefit from the temporarily lifting of barrier function in order to get access to highly protected compartments. In addition, some technologies further utilizes electrostatic gradient to allow a powered-mobilization of ionized drugs down the electrostatic gradient. Iontophoresis is a method used to enhance the delivery of charged molecules across tissue barriers that are relatively impermeable for ionized compounds. This rapid and noninvasive technique utilizes low electrical current to drive ionized molecules across barriers.

The main factors influencing iontophoretic drug delivery are the molecular weight, charge and lipophilicity of the drug, current density, the duration of treatment, pH and the permeability of the tissue. One electrode is placed on the eye while the other is placed on a remote tissue.

Therefore, ionic drugs are delivered from electrodes by current of the same polarity measured in milliamperes (mA). Thus, for negatively charged drugs, the negative pole (cathode) is placed on the eye and the positive pole (anode) on a remote location as the indifferent or ground electrode.

The total delivered electric charge is proportional to the delivered drug. The optimal setting that the operator should seek must allow for maximum delivery while minimizing the current level.

Thus, the efficiency of a iontophoretic device can be described in terms of the total amount of charge that must be passed to obtain the desired clinical effect (i.e. induction of local anesthesia upon delivery of lidocaine) and quantified in units of mA minutes, calculated by multiplying the electrical current and the application time.

Current (mA)×Application Time (min.)=Iontophoretic Dose (mA min.)

The efficiency of the iontophoresis process is expressed by the ratio:

[Micrograms of drug per gram (or ml) of target tissue]/[Iontophoretic Dose (mA min.)]

Proteins, peptides, oligonucleotides and plasmids are charged macromolecules that are relatively impermeable through ocular barriers and fit in the methodology of electrically-assisted penetration. Recently another electrically-assisted drug delivery technology, electroporation, was proposed as an alternative or adjuvant to iontophoresis. Electroporation comprises the use of electric pulses to induce transient changes in the cell membrane architecture that turn it into more permeable barrier. Beside the permeabilization effect on cell membrane, it was postulated that this technique induces electrophoretic effect on charged macromolecules and drives them to move across the destabilized membrane. These technologies, that their use is well known and readily available to those familiar with the art, are very useful for enhancing the penetration of biologically active drugs that possess many charged moieties, like the composition of the present invention.

“chemical penetration enhancers” in the present invention refers to the enhancement of drug penetration through tissue barriers by wide array of interventions that share the common ground of being able to disturb at least in part the barrier functions of that tissue and thus removing obstacles that limits drug penetration to the tissue. Preferably, the barrier effects of chemical penetration enhancers should be short lived to avoid consequences that can affect normal function of the tissue.

“biological penetration enhancers” in the present invention refers to the enhancement of drug penetration through a biological barriers by wide array of interventions that share the common ground of being able to favor the influx of the drug and minimize the efflux.

“tissue loosening agents” and “hyaluronidase and hyaluronidase analogs or equivalents” in the present invention refers to the use of agents to loosen up the surrounding extracellular matrix at the site of drug administration , aiming to significantly improve the distribution of the drug in ocular tissues and eventually enhancing its penetration. Although extensive experience have been gained with the use of hyaluronidase as tissue loosening agent, other analogs or equivalents of hyaluronidase could be utilized for the same purpose.

“delivery of said insulin PlGF and/or IGF is further assisted by antibody conjugated to” and “avidine-biotin or streptavidin-biotin system” in the present invention refers to the use of antibody assisted drug delivery wherein the antibody is attached to the drug delivery system by tailored linkers and the antibody selection should allow the specific targeting of the desired tissue. Methods to conjugate antibodies to liposomes, emulsions, polymeric drug delivery systems, and to naked proteins, peptides, plasmids and oligonucleotids, for site-specific targeting, are widely available for the skilled artisan. For example, by utilizing an efficiently internalized antibody, the conjugated drug, either naked or with a carrier, will be delivered inside targeted cells. (Wu A M, Senter P D, Nat Biotechnol. 2005; 23 (9): 1137-46), (Park J W, Benz C C, Martin F J, Semin Oncol. 2004; 31 (6 Suppl 13):196-205), (Hansen J E, Weisbart R H, Nishimura R N, Scientific world Journal. 2005; 5:782-8)

“pegylation” in the present invention refers to the process of covalent attachment of poly(ethylene glycol) polymer chains to another molecule, normally a drug or therapeutic protein. PEGylation is routinely achieved by incubation of a reactive derivative of PEG with the target macromolecule. The covalent attachment of PEG to a drug or therapeutic protein can “mask” the agent from the host's immune system increase the hydrodynamic size (size in solution) of the agent which prolongs its circulatory time by reducing renal clearance.

PEGylation can also provide water solubility to hydrophobic drugs and proteins.

“temporary punctal occlusion”, “permanent punctual occlusion”, “vascular constrictors”, “increased drug formulation adhesion to ocular tissues”, and “optimizing the volume of the administered drug formulation to prevent over spillage” in the present invention refers to methods and means to avoid systemic distribution of the locally administered active composition, in order to avoid unwanted side effects. Such methods are easily accessible and widely available to the skilled artisan. The widely accepted methods includes temporary punctal occlusion, permanent punctual occlusion, use of vascular constrictors, increased drug formulation adhesion to ocular tissues, and optimizing the volume of the administered drug formulation to prevent over spillage. (see: Abdulrazik M, Behar-Cohen F, Benita S. Drug Delivery Systems for Enhanced Ocular Absorption. In Enhancement in Drug Delivery, Touitou E, Barry B W, Eds., Taylor and Francis, pp. 489-526, 2006)

“cells that were converted into insulin PlGF and/or IGF-producing cells” in the present invention refers to converting cells that reside in-situ or those who can accumulate at the site of action of insulin PlGF and/or IGF, into insulin PlGF and/or IGF -producing cells.

“embryonic stem cells” and “adult stem cells” in the present invention refers to the utilizing of stem cells as insulin PlGF and/or IGF -producing cells. Stem cells are subdivided into embryonic stem cells and adult stem cells. Embryonic stem cells have pluripotent plasticity and can differentiate to any specialized cell line. They are derived from embryos that develop from eggs that have been fertilized by an in vitro fertilization process (IVF) and then donated for research purposes with informed consent of the donors. Adult stem cell is an undifferentiated cell found among differentiated cells in a tissue or organ, can renew itself and can differentiate to yield the major specialized cell types of the tissue or organ. Well known methods in the art teach ways of controlling stem cell differentiation in cell culture for specific purposes including cell-based therapies. Gene therapy applications that target stem cells offer great potential for the treatment of many kinds of diseases. However, a suitable gene delivery system should be tailored for each mission to meet the unique requirements and to overcome hurdles. For example, a plasmid-based system such as transfection methods combined with electroporation may be used to produce stable cell lines by selection to meet the target of mediating a long-term constitutive gene expression, while a transient expression system could utilize a cationic liposome-plasmid complex. Reportedly, some viral vectors, including some Adenovirus subtypes (e.g., fiber-modified Ad vectors, tropism-modified Ad5 vectors, and AdK7 vector), have shown favorable characteristics related to stem cell gene therapy like ease of vector preparation, high transduction efficiency, and the transient expression ability (See: Kawabata K, et al., Molecular Pharmaceutics, 2006; 3 (2):95-103).

In one examples of therapeutic utilization of stem cells, recent reported evidence have shown that it may be possible to direct the differentiation of human embryonic stem cells in cell culture to form insulin PlGF and/or IGF -producing cells that eventually could be used in transplantation therapy for diabetic patients. Another reported example includes replacing the dopamine-producing cells in the brains of Parkinson's patients.

See also: (Essentials of Stem Cell Biology, Lanza R. et al., Eds., Elsvier Academic Press, 2006),

(http://stemcells.nih.gov/info/basics/basics4.asp),
(http://www.news.wisc.edu/packages/stemcells/), (Lock L T, Tzanakakis E S. Tissue Eng. 2007; 13 (7):1399-412)

“viral gene delivery vector” and “non-viral gene delivery vector” in the present invention refers to assisted gene delivery process. These two categories differ primarily in their assembling process. While viral vector is assembled in a cell, nonviral vector is constructed at in vitro setting. Viral vectors are highly efficient in gene delivery. The desired therapeutic gene is incorporated in the viral coding sequence of replication-defective viruses. Viral vectors include adenovirus, adeno-associated virus, lentivirus, retrovirus, and herpes simplex virus, beside other less frequently used viruses.

Adenovirus is an icosahedral virus that contains a large DNA genome. Contrary to retrovirus, adenovirus can infect both dividing and nondividing cells. Gene expression using adenovirus lasts for only a short time because adenoviral genome is not integrated into the host DNA.

Adeno-associated virus (AAV) is a small nonpathogenic DNA virus. It is pathogenic only in the presence of a helper virus, such as adenovirus or herpes virus. In the absence of a helper virus, AAV instead incorporates its genome into the chromosomes of the host cells. Both dividing and non-dividing cells can be infected by AAV. The main disadvantage of AAV is that sufficiently high viral titer is hard to obtain. The virus also has a limited packaging size of about 5 kb.

Retrovirus is an eukaryotic RNA virus in the family Retroviridae that has RNA as its genome. It uses viral enzymes to copy its genome into DNA and integrate into the host chromosome.

Although recent advances in lentivirus vectors have enabled transduction of nondividing cells, long-term gene expression can he achieved with this vector only in the cycling cells.

Herpes simplex virus (HSV) is a double-stranded DNA virus with a genome measuring more than 150 kb. Similar to the advantages of adenovirus, HSV can infect a variety of dividing and nondividing cells. A very high viral titer can also he produced. However, HSV causes some cytotoxicity. The limitations of viral vectors, like insufficient pharmaceutical quantities (viral titers), toxicity, and the potential replication of competent viruses, make synthetic vectors an attractive alternative. Advantages of nonviral vectors include their nonimmunogenicity low acute toxicity, simplicity, and feasibility to be produced on a large scale. Still, nonviral vectors suffer from lower efficiency than viral vectors in gene transfer and their transient gene expressions.

Many approaches for gene therapy by nonviral vectors have been proposed including gene transfer with naked DNA. Gene transfer with naked DNA carries the advantages of its simplicity and lack of toxicity. It has been shown that simple injection of plasmid DNA directly into a tissue without additional help from either a physical force or a chemical agent is able to transfect cells. Gene transfer with naked DNA can be used for in vivo local injection of plasmid DNA into target ocular tissue to transfect selected ocular cells, or to transfect non-ocular cells and stem cells in vitro. Strategies to enhance naked DNA-mediated gene transfer will include the earlier described methods for protein stabilization, described elsewhere in the specifications, like the utilization of nuclease inhibitors. Other strategies to enhance naked DNA-mediated gene transfer will include the enhancement of DNA internalization by target cells through the use of hypotonic solution or the addition of surfactants, water-immiscible solvents, non-ionic polymers, and transferrin.

“physical gene delivery methods” in the present invention refers to the creation of transient injuries or defects on cell membranes that allows DNA to enter the cells by diffusion. Such methods include mechanical, electric (iontophoresis and electroporation), ultrasonic, hydrodynamic (hydrodynamic gene transfer), or laser energy. Shooting DNA into cells is one example that can be carried out by using a physical method such as bioballistic particle bombardment or gene gun. The gene gun uses gold particles coated with DNA and this approach is an ideal method for gene transfer to superficial or surgically exposed ocular tissues. DNA coated gold particles, which are accelerated by pressurized gas and expelled onto cells or a tissue. The momentum allows the gold particles to penetrate a few millimeters deep into a tissue and release DNA into cells on the path. Electrically assisted gene transfer is obviously invited as plasmids and oligonucleotides are charged macromolecules. Please consult the above mentioned description of the principles of iontophoresis and electroporation. Electroporation moves DNA along the electric field and can be practical in any ocular tissue into which a pair of electrodes can be inserted. Reportedly, the effective distance between the electrodes, is in the range of 1 cm.

Therefore, when targeting deep tissues, a surgical procedure is usually required to place the electrodes. Electroporation can be effective in delivering DNA as large as 100 kb. Overall, a short time interval between DNA administration and electroporation is beneficial to minimize DNA degradation by extracellular nucleases. Reportedly, a single electroporation assisted gene transfer can lead to a long-term gene expression. Ultrasound energy creates membrane pores and facilitates intracellular gene transfer through passive diffusion of DNA across the membrane pores. Ultrasound can facilitate gene transfer at cellular and tissue levels. The efficacy of ultrasound assisted transfection is determined by frequency, strength, and the duration of ultrasound treatment. The efficacy could be further increased by ultrasound enhancing agents that comprise air-filled microbubbles that rapidly expand and shrink under ultrasound irritation.

The consequent local shock waves cause transient changes in the permeability of neighboring cell membranes. Hydrodynamic gene delivery is based on brisk injection of naked plasmid DNA into selected body compartment at injected volume and injection pace that creates an overflow situation that transiently alters the permeability of the compartment barriers to the injected DNA, presumably by creation of transient membrane defects in neighboring cells. Thus, a direct gene transfer to the cytoplasm is achieved without endocytosis. Reportedly, this method can be effective in delivering DNA as large as 150 kb. In one embodiment of the present invention, gene transfer to the endothelium of schlemm's canal can be achieved by fast injection of DNA solution into the lumen of schlemm's canal. To enhance the efficacy of such intraluminal schlemm's canal injections, the injection could be accomplished by using a ballooned catheter and carrying out occlusion-assisted infusion to the whole or specific parts of the schlemm's canal. Treatment of ocular tissue with hyaluronidase or similar tissue loosening agents prior to injection of plasmid DNA to loosen up the surrounding extracellular matrix can significantly improve the distribution of plasmid DNA in the tissue and enhance transfection.

“cationic carriers for gene delivery” in the present invention refers to the utilization of cationic carriers for the formulation of DNA into condensed particles. Cell membrane is a major obstacle that faces DNA before getting access into the cytoplasm and migrating into the nucleus, where gene expression takes place. As DNA is an anionic macromolecule, cationic carriers are useful for the formulation of DNA into condensed particles. The reduced size of DNA-containing particles carries the obvious advantage of more efficient passage to the cytoplasm of target cells through the cell membrane via endocytosis, macropinocytosis, or phagocytosis. The charge ratio between the cationic carrier and the DNA should allow a net cationic charge of the DNA-carrier complex chat is sufficient for the induction of electrostatic interaction with anionic sites on cell surfaces to facilitate cellular uptake via endocytosis and similar mechanisms. DNA cationic carriers include cationic polymers (polyplexes), cationic lipids (lipoplexes), peptides, and lipid-polymer cationic hybrids. Cationic polymers that are effective candidates for the formation of polyplexes for gene delivery include cationic chitosan, poly-L-lysine, cationic dextran, polyallylamine, polypropylamine, polyamidoamine, and polyethylenimine. One sixth of the nitrogen atoms of polyethylenimine carry a positive charge at physiological pH, making it one of the most densely charged polymers. Reportedly, polyethylenimine is one of the most active polymers for gene delivery. Protein and peptide vectors for gene transfer into cells include cationic proteins and cationic peptides, like arginine-rich peptides derived from protamine, synthetic arginine-rich peptides, and lysine-rich peptides. Furthermore, Short cationic peptides can be utilized as membrane-penetrating molecules to enhance the efficiency of polyplex-mediated gene delivery.

“antibody mediated gene delivery” and “avidine-biotin or streptavidin-biotin” in the present invention refers to antibody assisted target-specific gene delivery. Conjugation to antibody or avidin can further provide the possibility of target-specific gene delivery.

See also: (Curtis AM, Ed., Viral vectors for gene therapy: methods and protocols, Humana Press, 2002), (Kazunari T, Kazunari K, Takuro N, Eds., Non-viral gene therapy: Gene design and delivery, Springer, 2005), (Gao X, Kim K S, Liu D. AAPS J. 2007; 9 (1):E92-104)

As the skilled artisan will appreciate, the aforementioned strategies may be used for introducing gene modifications to non-ocular, ocular, and periocular tissues, like cells adjacent to the schlemm's canal and lacrimal gland cells, as well as stem cells, to reach the target of regional or in-situ insulin, insulin, PlGF and/or IGF production by these gene modified cells on long- or short-term basis. The concentration of the insulin, PlGF and/or IGF composition which is supplied to the subject in need by means such as topical, corneal, intracameral, intravitreal should be sufficiently high to obtain the desired lowering of intraocular pressure. In order to achieve this, the concentration of insulin, PlGF and/or IGF in the administered composition is preferably between 10 and 1000 nmol/l more preferably between 50 and 500 nmol/l.

1. Use of a VEGF-B and combinations of VEGF-B and PLGF in the manufacture of a medicament for the treatment or prophylaxis of glaucoma and ocular hypertension.

2. A composition for the treatment or prophylaxis of glaucoma and ocular hypertension comprising an effective amount of VEGF-B, PLGF-1, PLGF-2, PLGF-3 or a combination thereof, that induces agonistic interaction with VEGFR-1, in a subject in need of such treatment.

3. A composition for the treatment or prophylaxis of glaucoma and ocular hypertension comprising the coadministration of an effective amount of VEGF-B, PLGF-1, PLGF-2, PLGF-3 or a combination thereof, with a compound selected from Insulin and Insulin like growth factor, and a pharmaceutically acceptable carrier suitable for injection into the eye or topical application to the eye, in a subject in need of such treatment.

4. A composition for the treatment or prophylaxis of glaucoma and ocular hypertension comprising the coadministration of an effective amount of VEGFR-1 agonist, a SPARC antagonist, and a compound selected from Insulin and Insulin like growth factor, and a pharmaceutically acceptable carrier suitable for injection into the eye or topical application to the eye, in a subject in need of such treatment.

5. The method of claim 1 wherein said VEGF-B is a VEGF-B polynucleotide or VEGF-B polypeptide, and wherein said PlGF is a PlGF polynucleotide or PlGF polypeptide.

6. The composition of claim 4 wherein said VEGFR-1 agonist is selected from VEGF-B, PlGF-1, PlGF-2, and PlGF-3.

7. The composition of claim 4 wherein said VEGFR-1 agonists, SPARC antagonists, insulin, insulin like growth factor, is their isoforms, their analogs, their gene-engineered modifications, and combinations thereof.

8. The method of claim 1 wherein said treatment or prophylaxis is for the prophylaxis, slowing, halting or reversal of ocular hypertension and/or glaucoma related damage to ocular tissues and cells.

9. The method of claim 1 wherein said treatment or prophylaxis is by increasing the success rate of glaucoma surgical procedures.

10. The method of claim 1 wherein said treatment or prophylaxis is by the prophylaxis, slowing, halting or reversal of damage to retinal ganglion cells.

11. The composition of claim 4 wherein said SPARC antagonist is an antibody to SPARC or antibody fragment that binds to SPARC.

12. The composition of claim 4 wherein said SPARC antagonist is siRNA that binds to and inhibits expression of the SPARC gene.

13. The composition of claim 4 wherein said SPARC antagonist is a peptide that binds to and inhibits the active site of SPARC.

14. The composition of claim 1 wherein said VEGF-B is VEGF-B.sub.167, and VEGF-B.sub.186.

15. The composition of claim 3 wherein said insulin or Insulin like growth factor is a euglycemic dose of insulin or Insulin like growth factor.

16. The composition of claim 1 wherein the composition administered route is selected from topical administration to the ocular surface, injection into the eye, controlled release device, ocular implant, intraocular implant, passive transscleral delivery, electrically assisted transscleral delivery, pump assisted transscleral delivery, regional administration, systemic administration.

17. The composition of claim 2 wherein the composition administered route is selected from topical administration to the ocular surface, injection into the eye, controlled release device, ocular implant, intraocular implant, passive transscleral delivery, electrically assisted transscleral delivery, pump assisted transscleral delivery, regional administration, systemic administration.

18. The composition of claim 3 wherein the composition administered route is selected from topical administration to the ocular surface, injection into the eye, controlled release device, ocular implant, intraocular implant, passive transscleral delivery, electrically assisted transscleral delivery, pump assisted transscleral delivery, regional administration, systemic administration.

19. The composition of claim 4 wherein the composition administered route is selected from topical administration to the ocular surface, injection into the eye, controlled release device, ocular implant, intraocular implant, passive transscleral delivery, electrically assisted transscleral delivery, pump assisted transscleral delivery, regional administration, systemic administration.

20. The method of claim 1 wherein said wherein said subject in need of such treatment is a human, human mammal, or pet.
